(19) United States
(12) Patent Application Publication
(10) Pub. No.: US 2018/0215391 A1
(43) Pub. Date: Aug. 2, 2018

(54) METHODS AND SYSTEMS FOR DETECTING ROAD SURFACE USING CROWD-SOURCED DRIVING BEHAVIORS

(71) Applicant: GM GLOBAL TECHNOLOGY OPERATIONS LLC, Detroit, MI (US)

(72) Inventors: JINZHU CHEN, TROY, MI (US); FAN BAI, ANN ARBOR, MI (US); QINGRONG ZHAO, MADISON HEIGHTS, MI (US); BAKHTIAR B. LITKOUHI, WASHINGTON, MI (US)

(73) Assignee: GM GLOBAL TECHNOLOGY OPERATIONS LLC, Detroit, MI (US)

(21) Appl. No.: 15/419,602

(22) Filed: Jan. 30, 2017

(51) Int. Cl. B60W 40/06; G07C 5/00; G01N 33/42

(52) U.S. Cl. CPC B60W 40/06; G07C 5/008; G01N 33/42; B60W 2540/30; B60W 2520/10; B60W 2550/40; B60W 2540/18; B60W 2540/10; B60W 2510/22; B60W 2750/40; B60W 2540/12

(57) ABSTRACT

Methods and systems are provided for determining a road surface condition. In one embodiment, a method includes: receiving vehicle data; constructing, by the processor, a driver behavioral model based on the vehicle data; determining, by the processor, a surface condition based on the driver behavioral model; and generating a signal based on the surface condition.

### TECHNICAL FIELD

The technical field generally relates to detecting road surface conditions, and more particularly relates to methods and systems that make use of crowd-sourced driving behaviors to detect a road surface condition and to control a vehicle based thereon.

### BACKGROUND

Road surface conditions can vary based on rain, snow, road anomalies such as potholes, road debris, bumps, and slippery road, etc. An accurate detection of a slippery road surface or potentially hazardous pavement conditions improves vehicle control, improves route determination, among other benefits. Accurate information about such road conditions is generally not available.

Drivers may substantially change their driving behavior when a road surface is slippery. For example, when a road is slippery, a driver may apply the brakes and accelerate more moderately. The behavior of other drivers can be detected by a vehicle using one or more sensors of the vehicle and/or be communicated between vehicles and between vehicles and infrastructure.

Accordingly, it is desirable to provide methods and systems for determining road surface condition based on other drivers' behaviors. Furthermore, other desirable features and characteristics of the present invention will become apparent from the subsequent detailed description and the appended claims, taken in conjunction with the accompanying drawings and the foregoing technical field and background.

### SUMMARY

Methods and systems are provided for determining a road surface condition and controlling a feature of the vehicle based thereon. In one embodiment, a method includes: receiving vehicle data; constructing, by the processor, a driver behavioral model based on the vehicle data; determining, by the processor, a surface condition based on the driver behavioral model; and generating a signal based on the surface condition.

In one embodiment, a system includes: a non-transitory computer readable medium. The non-transitory computer readable medium includes a first module configured to, by a processor, receive vehicle data and construct a driver behavioral model based on the vehicle data. The non-transitory computer readable medium further includes a second module configured to, by a processor, determine a surface condition based on the driver behavioral model. The non-transitory computer readable medium further includes a third module configured to, by a processor, generate a signal based on the surface condition.

### DESCRIPTION OF THE DRAWINGS

The exemplary embodiments will hereinafter be described in conjunction with the following drawing figures, wherein like numerals denote like elements, and wherein:

FIG. 1 is a functional block diagram of a vehicle having a road surface determination module in accordance with various embodiments;

FIG. 2 is a dataflow diagram illustrating a road surface determination module in accordance with various embodiments; and

FIGS. 3 and 4 are flowcharts illustrating methods of determining road surface condition in accordance with various embodiments.

### DETAILED DESCRIPTION

The following detailed description is merely exemplary in nature and is not intended to limit the application and uses. Furthermore, there is no intention to be bound by any expressed or implied theory presented in the preceding technical field, background, brief summary or the following detailed description. As used herein, the term module refers to an application specific integrated circuit (ASIC), an electronic circuit, a processor (shared, dedicated, or group) and memory that executes one or more software or firmware programs, a combinational logic circuit, and/or other suitable components that provide the described functionality.

Embodiments of the invention may be described herein in terms of functional and/or logical block components and various processing steps. It should be appreciated that such block components may be realized by any number of hardware, software, and/or firmware components configured to perform the specified functions. For example, an embodiment of the invention may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that embodiments of the present invention may be practiced in conjunction with any number of control systems, and that the vehicle system described herein is merely one example embodiment of the invention.

For the sake of brevity, conventional techniques related to signal processing, data transmission, signaling, control, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in an embodiment of the invention.

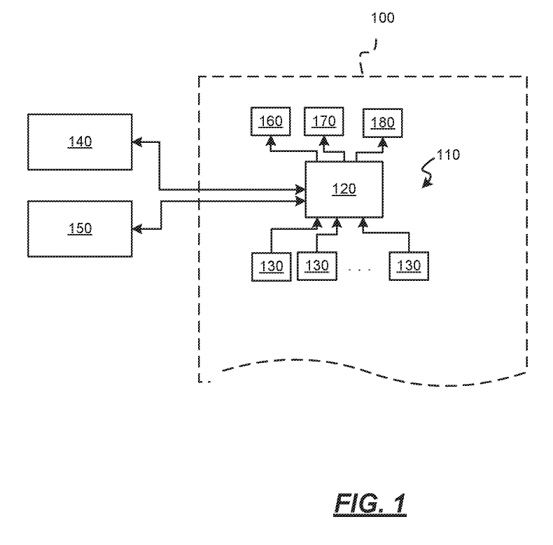

With reference to FIG. 1, an exemplary vehicle 100 in part that includes a control system 110 is shown in accordance with exemplary embodiments. As can be appreciated, the vehicle 100 may be any vehicle type that travels over a road surface. Although the figures shown herein depict an example with certain arrangements of elements, additional intervening elements, devices, features, or components may be present in actual embodiments. It should also be understood that FIG. 1 is merely illustrative and may not be drawn to scale.

The control system 110 includes a control module 120. As can be appreciated, the control module 120 can reside entirely on the vehicle 100, can reside partially on the vehicle 100 and reside partially on a remote server (not shown), and/or can reside entirely on a remote server (not shown). For exemplary purposes, the disclosure will be discussed in the context of the control module 120 residing entirely on the vehicle 100.

The control module 120 receives inputs from one or more sensors 130 of the vehicle 100. The sensors 130 sense observable conditions of the vehicle 100 and generate sensor signals based thereon. For example, the sensors 130 sense a vehicle speed, vehicle acceleration, a brake pedal position, an accelerator pedal position, a steering wheel angle, a vehicle suspension state, a rate of change of any of the values, and/or other vehicle system values and generate sensor signals based thereon. In various embodiments, the sensors 130 communicate the signals directly to the control module 120 and/or may communicate the signals to other control modules (not shown) which, in turn, communicate data from the signals to the control module 120 over a communication bus (not shown) or other communication means. In various embodiments, the control module receives signals from or sends signals to other vehicles 140 or infrastructure 150 via a wireless communication system.

The control module 120 receives the signals and/or the data captured by the sensors 130 and the wirelessly communicated signals communicated by the other vehicles 140 and/or infrastructure 150 and determines a road surface condition based thereon. In various embodiments, the control module 120 determines the road surface condition to be dry or slippery as discussed herein. As can be appreciated, the control module 120 can determine the road surface condition to have other characteristics such as, but not limited to, potholes, debris, bumps or other road conditions in various embodiments. Thus, the disclosure is not limited to the exemplary embodiments referring to dry or slippery road surface conditions.

The control module 120 receives the signals and/or the data captured by the sensors 130 and determines a local road surface condition. The control module 120 then receives other road surface conditions determined by the other vehicles 140 (i.e. crowd-sourced road surface conditions) and determines an overall road surface condition based on an analysis of the local road surface condition and the other road surface conditions. The control module 120 then uses the overall road surface condition to control one or more feature systems 160 of the vehicle 100, to generate notifications to the driver or other drivers via a notification system 170, and/or to re-route navigation of the vehicle 100 or other vehicles 140 via a navigation system 180.

For example, the control module 120 can determine the road surface condition to be a slippery surface, and based thereon: the control module 120 determines an autonomous actuating vehicle braking strategy; communicates the road surface condition to a wireless communication system for alerting other vehicle drivers of the identified slippery surface; alerts a driver of a potential reduced traction between vehicle tires and the surface as a result of the slippery surface; alerts a driver to not use a driver assistance system; and/or provides a notification of the slippery surface to a vehicle controller (not shown), and the vehicle controller autonomously modifies a control setting of an automated control feature in response to the notification. As can be appreciated, the overall road surface condition can be used by other vehicle features and is not limited to the present examples.

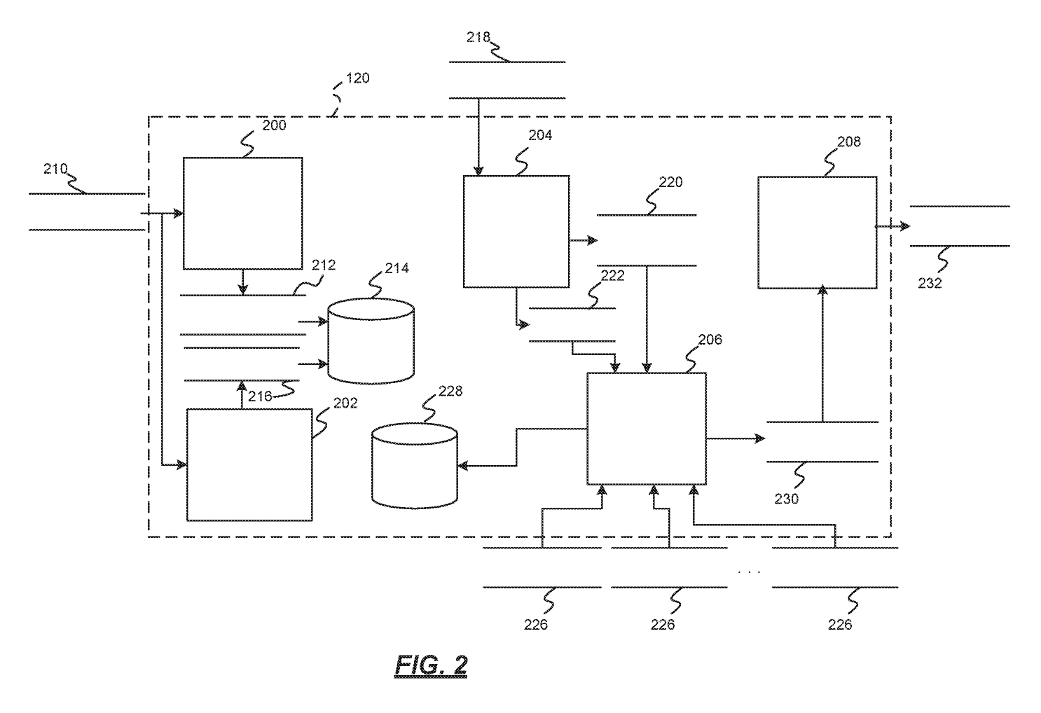

Referring now to FIG. 2 and with continued reference to FIG. 1, a dataflow diagram illustrates the control module 120 in accordance with various exemplary embodiments. As can be appreciated, various exemplary embodiments of the control module 120, according to the present disclosure, may include any number of sub-modules. In various exemplary embodiments, the sub-modules shown in FIG. 2 may be combined and/or further partitioned to similarly determine a road surface condition and to control the vehicle 100 based thereon. In various embodiments, the control module 120 includes a driving behavior learning module 200, a model quality determination module 202, a local road surface condition determination module 204, crowd-based road surface condition determination module 206, and at least one feature control/notification module 208.

The driving behavior learning module 200 receives vehicle data 210 such as, but not limited to, vehicle speed data, brake pedal position data, accelerator pedal position data, suspension system data, and steering wheel angle data. Based on the vehicle data 210, the driving behavior learning module 200 learns driving behavior of the vehicle 100 over time and constructs driver behavior models 212 based on the driving behaviors. For example, the driver behavior models 212 can be constructed based on two exemplary surface conditions dry (H₀) and slippery (H₁). For each surface condition, driver behavior models 212 are constructed using the vehicle data 210 for various vehicle speed ranges.

For example, provided the probabilities P(H₀|X, V) and P(H₁|X, V), where X represents vehicle data (i.e., brake pedal position data, accelerator pedal position data, steering wheel angle data, etc.) and V represents vehicle speed. The vehicle speed (V) is divided into (N) bins or ranges (Vₙ<Vₙ₊₁≤Vₙ₊₂ (n∈[1, N])), and the probability of vehicle conditions for each vehicle speed bin or range and surface condition represented as i={0,1} are provided as:

$$P ( {H}_{i} \vert X , {V}_{n} ) = \frac{P ( X , {V}_{n} , {H}_{i} )}{P ( X , {V}_{n} )} = \frac{P ( X \vert {V}_{n} , {H}_{i} ) P ( {V}_{n} \vert {H}_{i} ) P ( {H}_{i} )}{P ( X \vert {V}_{n} ) P ( {V}_{n} )} .$$

Where P(XI Vₙ, Hᵢ) represents the probability of X under speed Vn over a dry or a slippery dataset; P(Vₙ|Hᵢ) represents the probability of a speed bin over a dry or a slippery dataset, per segment; and P(Hᵢ) represents the prior predicted probability of being over a road surface condition (e.g., dry or slippery surface). As can be appreciated, the segment refers to a road segment. The road segment can be static (defined in advance) or dynamic (real-time). The probability for each bin P(X|Vₙ, Hᵢ) is then calibrated based on potential clustering. The driver behavior models 212 are stored in a datastore 214 for future use.

The model quality determination module 202 receives the vehicle data 210 and determines a quality 216 of each of the driver behavior models 212. For example, the model quality determination module 202 determines the quality 216 based on how significantly the driver changes his behavior. The significance in the change can be measured by a distance measurement (D_{XV}) between P(H₀|X, V) and P(H₁|X, V). For example, a Bhattacharyya or other distance function may be used.

If

$$P ( X \vert {V}_{n} , {H}_{0} ) \sim ( {μ}_{0} , {σ}_{0}^{2} ) , P ( X \vert {V}_{n} , {H}_{1} ) \sim ( {μ}_{1} , {σ}_{1}^{2} )$$

$${D}_{{XV}_{n}} = \frac{1}{4} ln ( \frac{1}{4} ( \frac{{σ}_{1}^{2}}{{σ}_{2}^{2}} + \frac{{σ}_{2}^{2}}{{σ}_{1}^{2}} + 2 ) ) + \frac{1}{4} ( \frac{{( {μ}_{1} - {μ}_{2} )}^{2}}{{σ}_{1} + {σ}_{2}} )$$

D_{XV}_{ₙ} of non-normal distributions can be calculated from integration. The model quality determination module 202 stores the model quality 216 for each of the models in the datastore 214 for future use.

The local road surface condition determination module 204 receives vehicle data 218 such as, but not limited to, speed data, brake pedal position data, accelerator pedal position data, suspension data, and steering wheel angle data. The local road surface condition determination module 204 determines a local road surface condition 220 by evaluating the vehicle data 218 using a learned driver behavior model 212 stored in the datastore 214. The learned driver behavior model 211 observes a set of derived indicators of different driving behaviors among different road conditions. The local road surface condition determination module 204 makes available the local road surface condition 220 and the quality 222 of the driver behavior model 212 used to determine the local road surface condition 220.

The crowd-based road surface condition determination module 206 receives the local road surface condition 220 and the quality 222 from the vehicle 100, and the local road surface condition information 226 (including the quality of the type, a road segment identifier, and/or a vehicle identification) from the other vehicles 140. The crowd-based road surface condition determination module 206 compiles the local road surface condition information 220, 222, and 226 based on the associated road segment and the vehicle ID and stores the compiled data in a datastore 228. The crowd-based road surface condition determination module 206 determines an overall road surface condition 230 based on the compiled data. For example, for each road segment having sufficient data stored in the datastore 228, the crowd-based road surface condition determination module 206 selects the data from N vehicles and determines whether the road type is of a certain quality (for example, dry: 1 or not dry: 0) based on the following relationship:

IF

$${\sum }_{i = 1}^{N} ( \frac{{w}_{i}}{{\sum }_{i = 1}^{N} {w}_{i}} {S}_{i} {e}^{- α {t}_{i}} ) > {φ}_{cloud}$$

THEN road surface condition is Si.

Where N represents the number of selected vehicles; Sᵢ represents the local road surface condition decision; wᵢ represents the driver behavior model quality; tᵢ represents the time elapsed since the local detection; α represents the decay coefficient; ϕ_{cloud} represents the decision threshold.

The at least one feature control/notification module 208 receives as input the overall surface condition 230. The feature control/notification module 208 generates signals 232 to control one or more features of the vehicle 100 or other vehicles 140, to generate notifications to the driver or other drivers, and/or to re-route navigation. In various embodiments, the feature control/notification module 208 provides relevant information to infrastructure 150.

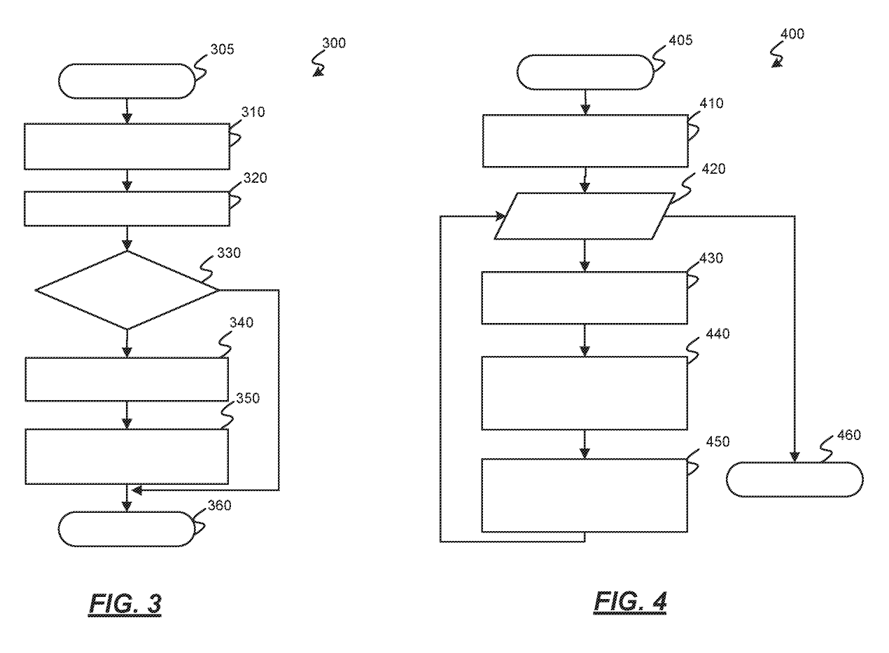

With reference now to FIGS. 3 and 4, and with continued reference to FIGS. 1-2, flowcharts are shown of methods 300 and 400 for determining a road surface condition and controlling a vehicle based thereon, in accordance with various embodiments. The methods 300 and 400 can be implemented in connection with the vehicle 100 of FIG. 1 and can be performed by the control module 120 of FIGS. 1 and 2, in accordance with various exemplary embodiments. As can be appreciated in light of the disclosure, the order of operation within the method is not limited to the sequential execution as illustrated in FIGS. 3 and 4, but may be performed in one or more varying orders as applicable and in accordance with the present disclosure. As can further be appreciated, the methods of FIGS. 3 and 4 may be scheduled to run at predetermined time intervals during operation of the vehicle 100 and/or may be scheduled to run based on predetermined events.

In various embodiments, the method 300 may be performed to determine the local road surface condition 220, for example, by the control module 120. In one example, the method 300 may begin at 305. The driver behavior models 212 are constructed at 310 for example, as discussed above. The model quality is determined at 315 and evaluated at 320. If the model quality is sufficient (e.g., greater than a defined threshold) at 330, the local road surface condition 220 is determined based on the driver behavior models 212 and the vehicle data 218 at 340, for example as discussed above, and communicated for use along with, for example, the quality 222, the corresponding road segment, the vehicle identification, the time, etc. at 350. Thereafter, the method may end at 360.

In various embodiments, the method 400 may be performed to determine the overall surface condition 230, for example, by a server system or the control module 120. In one example, the method 400 may begin at 405. The local road surface condition information 220, 222, 226 is received from any number of vehicles 100, 140 and compiled at 410. For each road segment having sufficient data at 420, the best vehicles (N) that provided local road surface condition information 220, 222, 226 for the segment are selected at 430. The cloud decision is made based on the local road types from the number N vehicles at 440 for example as discussed above and communicated to the vehicles 100, 140 (e.g., based on a request, a location, or other information) at 450. The method continues until the road segments have been processed at 420 and the method ends at 460.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the disclosure in any way. Rather, the foregoing detailed description will provide those skilled in the art with a convenient road map for implementing the exemplary embodiment or exemplary embodiments. It should be understood that various changes can be made in the function and arrangement of elements without departing from the scope of the disclosure as set forth in the appended claims and the legal equivalents thereof.

What is claimed is:

1. A method for detecting road surface condition, comprising:
receiving vehicle data;
constructing, by the processor, a driver behavioral model based on the vehicle data;
determining, by the processor, a surface condition based on the driver behavioral model; and
generating a signal based on the surface condition.

2. The method of claim 1, further comprising compiling the determined surface conditions from a plurality of vehicles, and determining an overall surface condition based on the compiled surface conditions.

3. The method of claim 2, wherein the determining the overall surface condition is based on a weighted voting method.

4. The method of claim 2, wherein the determining the overall surface condition comprises selecting N surface conditions based on a significance in a change of driving behavior.

5. The method of claim 4, further comprising determining a quality of the driver behavioral model based on the significance in change of the driving behavior and a comparison to a default driving behavior and wherein the selecting the N surface conditions is based on the quality.

6. The method of claim 1, wherein the surface condition is determined to be at least one of dry and slippery.

7. The method of claim 1, wherein the vehicle data includes at least one of vehicle speed, acceleration behavior, braking behavior, steering behavior, and suspension state.

8. The method of claim 1, further comprising constructing a plurality of driver behavioral models based on vehicle speed.

9. The method of claim 8, further comprising constructing the plurality of behavioral models based on vehicle braking behaviors and vehicle acceleration behaviors.

10. The method of claim 1, wherein the signal is a communication signal to a remote server.

11. A system, comprising:
a non-transitory computer readable medium, comprising:
a first module configured to, by a processor, receive vehicle data and construct a driver behavioral model based on the vehicle data;
a second module configured to, by a processor, determine a surface condition based on the driver behavioral model; and
a third module configured to, by a processor, generate a signal based on the surface condition.

12. The system of claim 11, further comprising a fourth module configured to compile the determined surface conditions from a plurality of vehicles, and determine an overall surface condition based on the compiled surface conditions.

13. The system of claim 12, wherein the further module determines the overall surface condition based on a weighted voting method.

14. The system of claim 12, wherein the fourth module determines the overall surface condition by selecting N surface conditions based on a significance in a change of driving behavior.

15. The system of claim 14, further comprising a fifth module that determines a quality of the driver behavioral model based on the significance in change of the driving behavior and wherein the fourth module selects the N surface conditions based on the quality.

16. The system of claim 11, wherein the surface condition is determined to be at least one of dry and slippery.

17. The system of claim 11, wherein the vehicle data includes at least one of vehicle speed, acceleration behavior, braking behavior, suspension state, and steering behavior.

18. The system of claim 11, wherein the first module is configured to construct a plurality of driver behavioral models based on vehicle speed.

19. The system of claim 18, wherein the first module is configured to construct the plurality of behavioral models based on vehicle braking behaviors and vehicle acceleration behaviors.

20. The system of claim 11, wherein the signal is a communication signal communicated to a remote server.
